(19) United States
(12) Patent Application Publication
(10) Pub. No.: US 2013/0103016 A1
(43) Pub. Date: Apr. 25, 2013

(54) AUTOMATIC BEAUTY THERAPY EQUIPMENT

(71) Applicants: Yi-San CHANG, Tainan (TW); Che-Chang Hsu, Tainan (TW); Shyang-Jye Chang, New Taipei (TW); Sheau-Chiou Chao, Tainan (TW)

(72) Inventors: Yi-San CHANG, Tainan (TW); Che-Chang Hsu, Tainan (TW); Shyang-Jye Chang, New Taipei (TW); Sheau-Chiou Chao, Tainan (TW)

(21) Appl. No.: 13/329,221

(22) Filed: Dec. 17, 2011

(30) Foreign Application Priority Data
Oct. 21, 2011 (TW) 100138311

(51) Int. Cl. A61B 18/18; A61B 18/20

(52) U.S. Cl. 606/9

(57) ABSTRACT

An automatic beauty therapy equipment contains a displacement driving device including a pedestal to be driven by a displacing mechanism so as to move straightly or curvedly in a first-axis direction, a second-axis direction, and a third-axis direction; a therapy device including a therapy element fixed in the pedestal; a controlling device including a controller electrically connecting with the displacement diving device and the therapy device, the controller being used to control the displacement driving device to urge the therapy element of the therapy device to move straightly or curvedly within a therapy area in the first-axis, the second-axis, and the third-axis directions, and the controller being served to set a therapy area so that the therapy element is driven to move vertically and swing with a curved profile of the therapy area to have a precise and stable beauty therapy in the therapy area.

### BACKGROUND OF THE INVENTION

1. Field of the Invention

The present invention relates to automatic beauty therapy equipment in which the therapy element can be operated precisely and stably in the therapy area to achieve a therapy effect well.

2. Description of the Prior Arts

As shown in FIG. 1, a conventional manual therapy element 10 is operated manually to move within a therapy area 11 to irradiate laser energy on skin to have a beauty therapy. However, such a manual operation still has the following defects:

1. When moving therapy element 10 manually, it has to be keep a suitable distance from the therapy area 11, or a beauty therapy effect can not be obtained as the distance between the therapy element 10 and the therapy area 11 is large; yet a burning will happen as distance between the therapy element 10 and the therapy area 11 is small.

2. Because the therapy element is operated manually, a fatigue will generate while operating in a long time.

3. The distance between the therapy element 10 and the therapy area 11 can not be controlled precisely due to a manual operation. Bedsides, the beauty therapy in a large area can not be achieved because of the manual operation.

The present invention has arisen to mitigate and/or obviate the afore-described disadvantages.

### SUMMARY OF THE INVENTION

The primary object of the present invention is to provide an automatic beauty therapy equipment in which the controller controls the therapy element of the pedestal of the displacement driving device to move straightly or curvedly in the first-axis, the second-axis, and the third-axis directions within the therapy area based on a set therapy parameter, the sensed distance, and the sensed temperature, and during a beauty therapy course, the therapy element is driven to move vertically and swing with a curved profile of the therapy area.

Further object of the present invention is to provide an automatic beauty therapy equipment in which the controller is used to set therapy parameter so that the therapy element moves vertically and swing with the curved profile of the therapy area and keeps a suitable therapy distance from the therapy area to have a precise and stable beauty therapy in the therapy area.

Another object of the present invention is to provide an automatic beauty therapy equipment in which the therapy element keeps a suitable therapy distance from the therapy area to have a precise and stable beauty therapy in the therapy area to prevent from a manual operation.

Also another object of the present invention is to provide an automatic beauty therapy equipment in which the left slideway and the right sideway are positioned above different sizes of therapy areas to obtain precise beauty therapy.

### BRIEF DESCRIPTION OF THE DRAWINGS

FIG. 1 is a perspective view of a conventional manual therapy element;

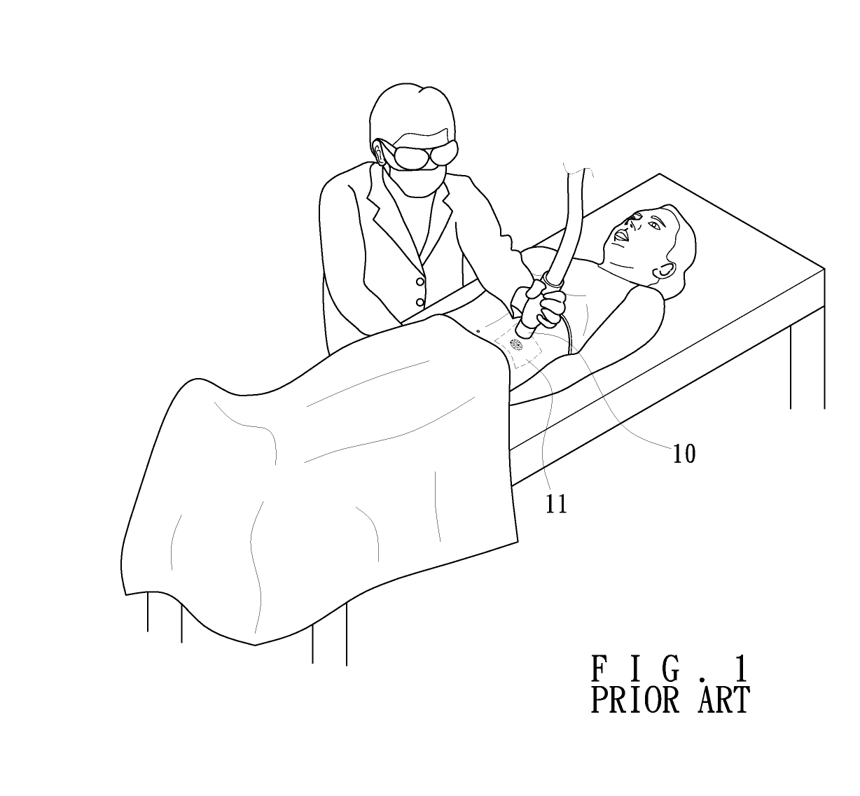

FIG. 2 is a perspective view showing the assembly of an automatic beauty therapy equipment according to a preferred embodiment of the present invention;

FIG. 3 is a plan view showing the assembly of the automatic beauty therapy equipment according to the preferred embodiment of the present invention;

FIG. 4 is a cross sectional view taken along the lines A-A of FIG. 3;

FIG. 5 is a plan view taken along the lines B-B of FIG. 4;

FIG. 6 is a schematic view showing the operation of the automatic beauty therapy equipment according to the preferred embodiment of the present invention;

FIG. 7 is a plan view showing the operation of the automatic beauty therapy equipment according to the preferred embodiment of the present invention;

FIG. 8 is a perspective view showing the operation of the automatic beauty therapy equipment according to the preferred embodiment of the present invention;

FIG. 9 is another schematic view showing the operation of the automatic beauty therapy equipment according to the preferred embodiment of the present invention;

FIG. 10 is another perspective view showing the operation of the automatic beauty therapy equipment according to the preferred embodiment of the present invention;

FIG. 11 is also another perspective view showing the operation of the automatic beauty therapy equipment according to the preferred embodiment of the present invention;

FIG. 12 is also another schematic view showing the operation of the automatic beauty therapy equipment according to the preferred embodiment of the present invention; and

FIG. 13 is another perspective view showing the operation of the automatic beauty therapy equipment according to the preferred embodiment of the present invention.

### DETAILED DESCRIPTION OF THE PREFERRED EMBODIMENT

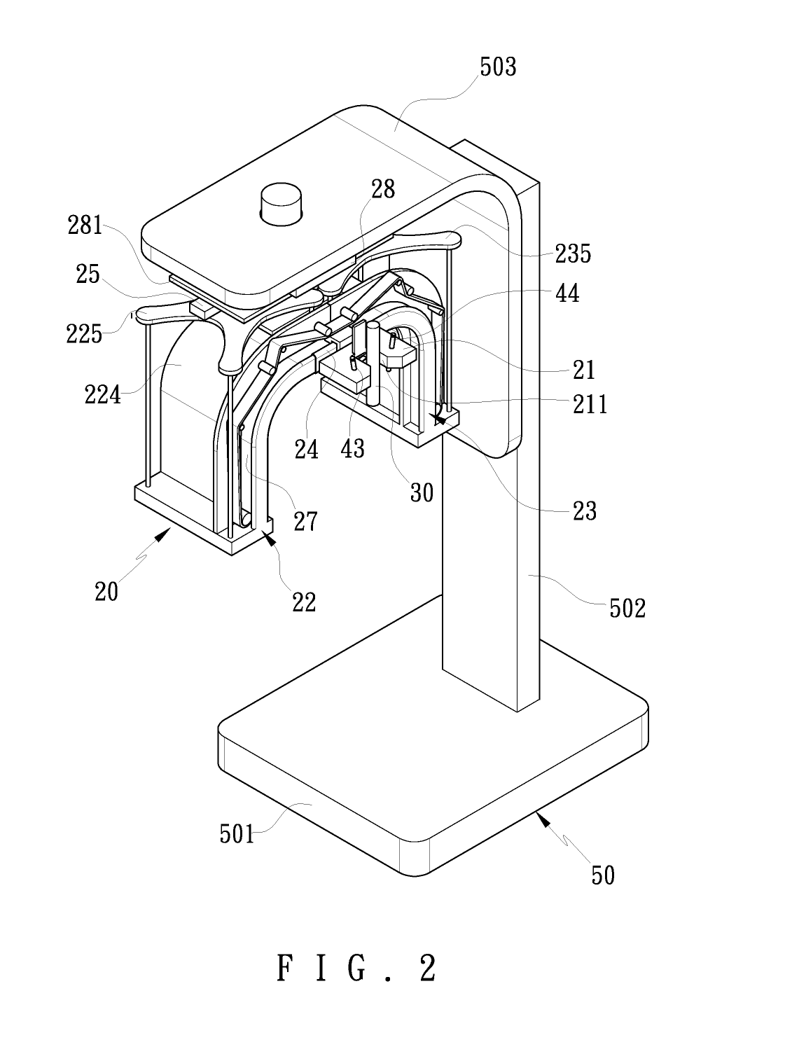

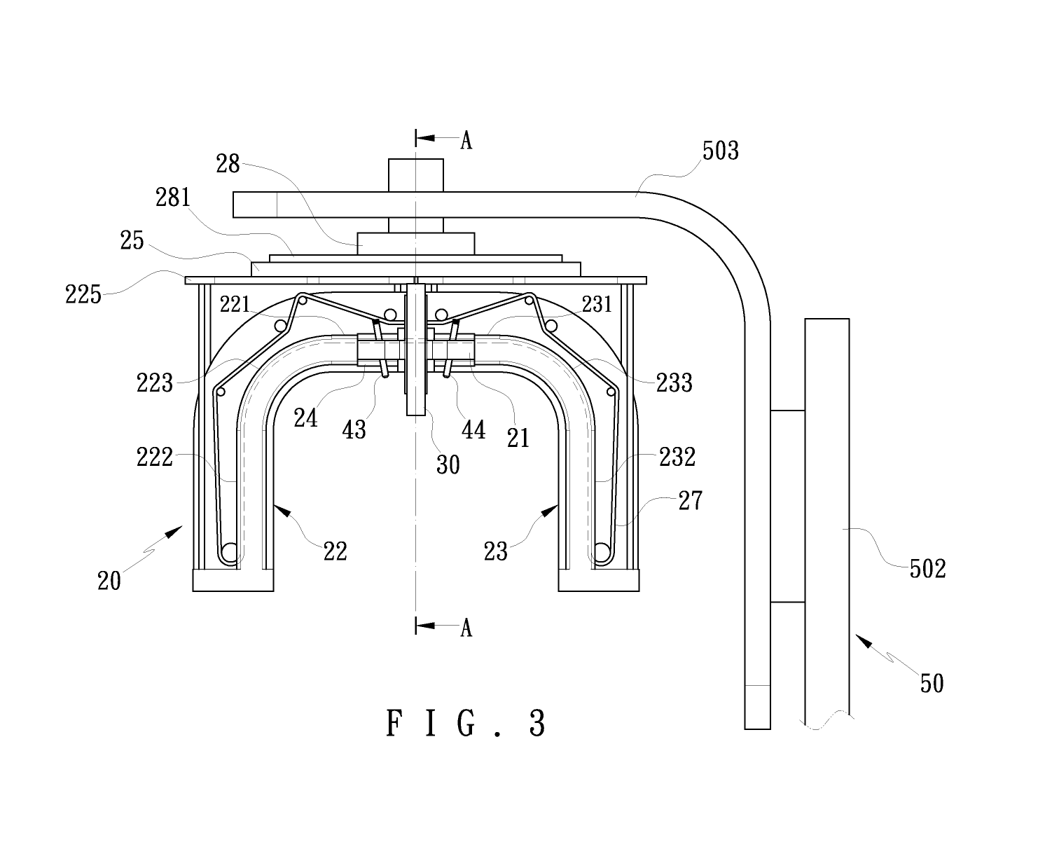

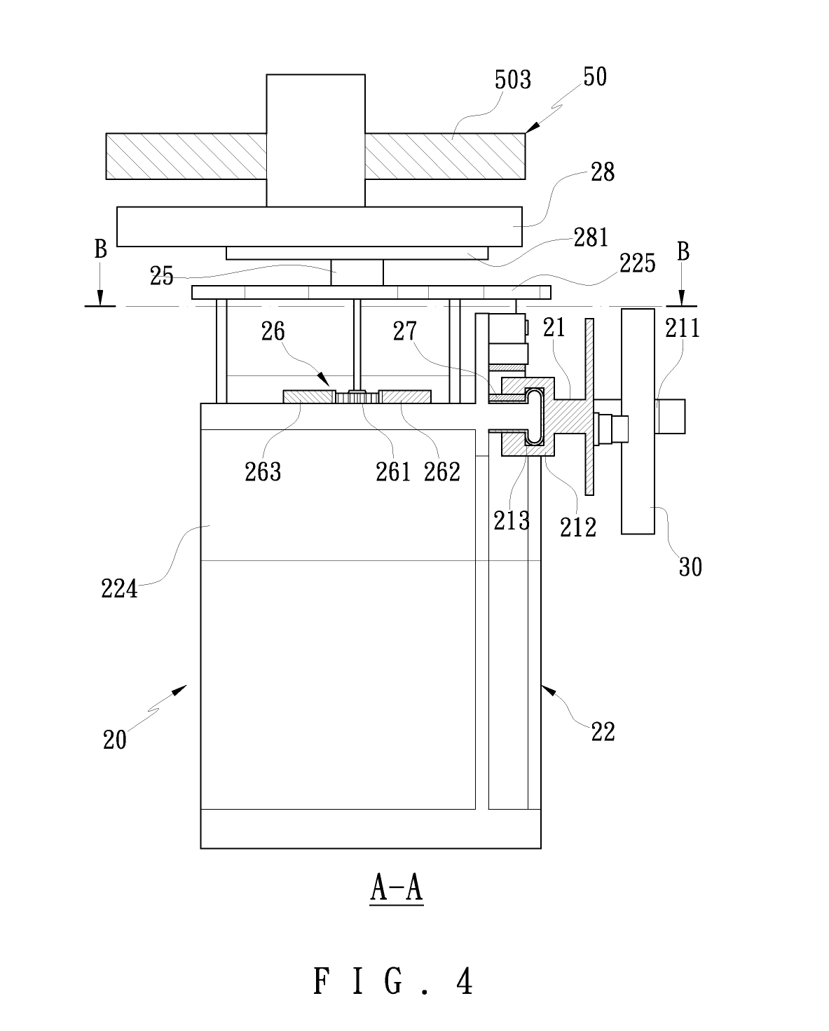

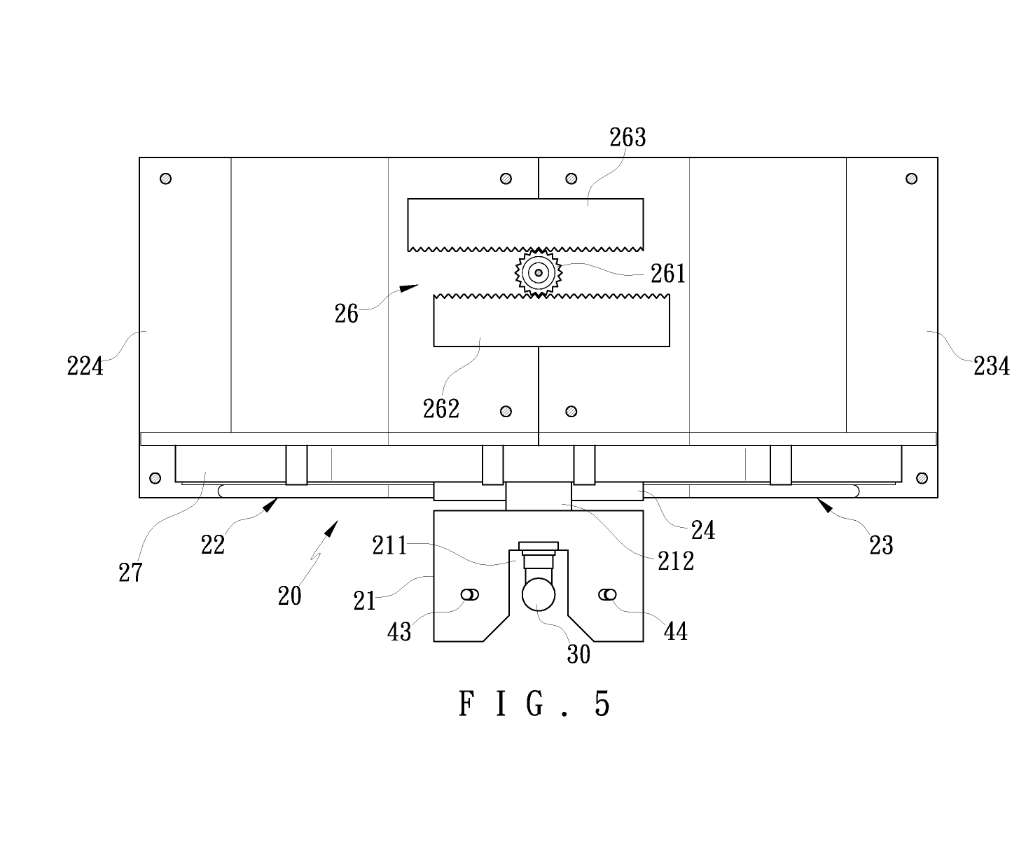

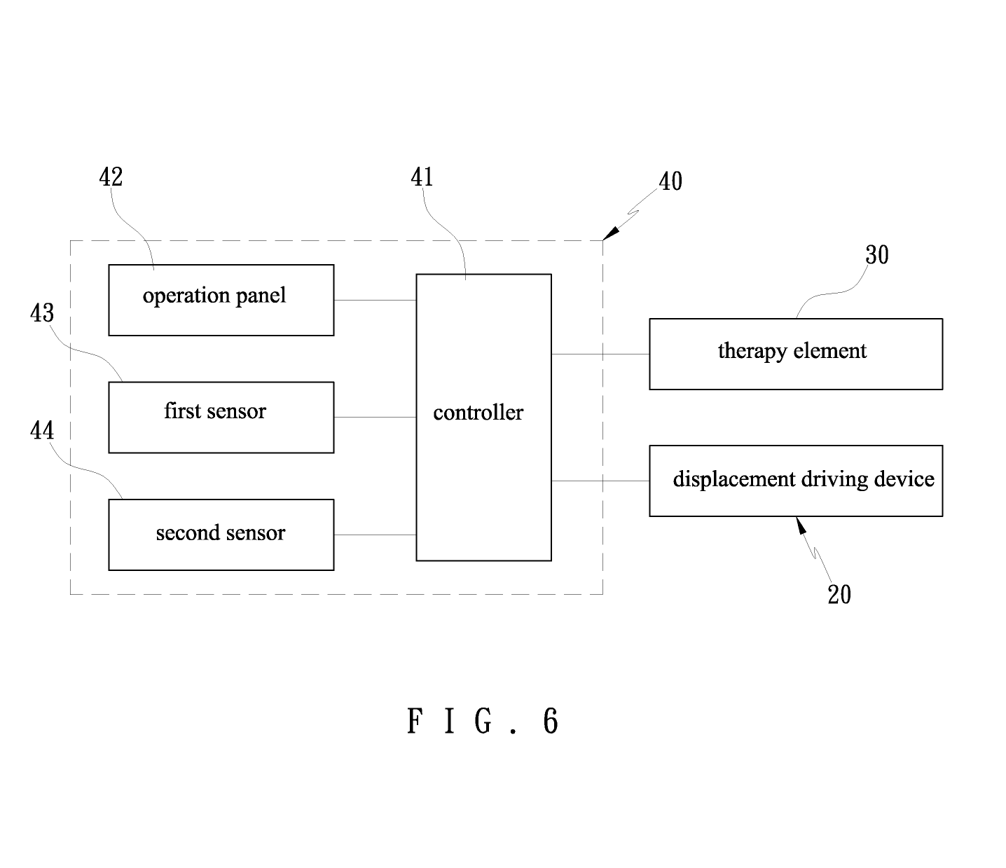

Referring to FIGS. 2-6, an automatic beauty therapy equipment according to a preferred embodiment of the present invention comprises a displacement driving device 20, a therapy device, a controlling device 40, and a supporting member 50; the displacement driving device 20 includes a pedestal 21 to be driven by a displacing mechanism so as to move axially, and the pedestal 21 includes a receiving portion 211 disposed on one side thereof and an actuating block 212 mounted on another side thereof and connected onto the displacing mechanism so that the pedestal 21 moves axially; in this embodiment, the displacing mechanism includes a first guiding set and a second guiding set, wherein the first guiding set is coupled with the pedestal 21 and formed in a door shape to drive the pedestal 21 to displace straightly or curvedly in a first-axis direction and a second-axis direction, the second guiding set is connected with the first guiding set to drive the pedestal 21 to move straightly in a third-axis direction; wherein the first guiding set includes a left slideway 22 and a right sideway 23, both of which include a first horizontal section 221 and a second horizontal section 231 extending in a first-axis direction respectively and a first vertical section 222 and a second vertical section 232 extending in the second-axis direction individually, the first horizontal section 221 connects with the second horizontal section 231, and between the first horizontal section 221 and the first vertical section 222 is defined a first arcuate section 223, between the second horizontal section 231 and the second vertical section 232 is defined a second arcuate section 233, the left slideway 22 includes a first side plate 224 extending from one side thereof, the right slideway 23 includes a second side plate 234 extending from one side thereof, a first top plate 225 is connected with the first side plate 224, a second side plate 235 is coupled with the second side plate 234. It is preferable that between the left slideway 22 and the right slideway 23 is defined an adjusting structure to adjust an interval width between the left slideway 22 and the right slideway 23 so that the left slideway 22 and the right slideway 23 are positioned above a therapy area; in this embodiment, the adjusting structure includes a connecting rail 24 fixed on a connection of the first horizontal section 221 of the left slideway 22 and the second horizontal section 231 of the right sideway 23, and one end of the connecting rail 24 is connected with the first horizontal section 221 of the left slideway 22, another end of the connecting rail 24 is coupled with the second horizontal section 231 of the right slideway 23, the first top plate 225 of the left slideway 22 and the second plate 235 of the right sideway 23 are joined with a slidable rail assembly 25 so that the left slideway 22 and the right sideway 23 allows for a movement in the first-axis direction, and between the first side plate 224 of the left slideway 22 and the second side plate 234 of the right slideway 23 is defined an actuation assembly 26 in which a gear 261 is fixed to be driven to rotate, two sides of the gear 261 engage with a first rack 262 of the first side plate 224 and a second rack 263 of the second side plate 234 respectively to drive the first rack 262 and the second rack 263 individually so that the first rack 262 and the second rack 263 actuate the left slideway 22 and the right slideway 23 to displace in the first-axis direction respectively, the connecting rail 24 is fitted with the first horizontal section 221 of the left slideway 22 and the second horizontal section 231 of the right sideway 23 to form a displacement gap by which the interval width between the left slideway 22 and the right slideway 23 is adjusted; a groove 213 of the pedestal 21 is mounted on the left slideway 22, the right slideway 23, and the connecting rail 24, and a belt 27 is driven to rotate along the left slideway 22, the right slideway 23, and the connecting rail 24 so as to actuate the actuating block 212 of the pedestal 21, such that the pedestal 21 is driven to displace straightly or curvedly along the left slideway 22 and the right slideway 23 in the first-axis direction and the second-axis direction individually; the second guiding set of the displacing mechanism includes a vertical slideway 28 extending therefrom in the third-axis direction, and the vertical slideway 28 includes a seat 281 disposed on a bottom end thereof to connect with the slidable rail assembly 25 of the adjusting structure so that the left slideway 22 and the right slideway 23 are driven to displace straightly in the third-axis direction, and the pedestal 21 is actuated by the left slideway 22 and the right slideway 23 to displace in the third-axis direction; the first guiding set and the second guiding set respectively includes a driving unit (not shown), such as a motor, to urge the pedestal 21 to slide along the first guiding set and to drive the first guiding set to slide along the second guiding set; the therapy device includes a therapy element 30 fixed in the receiving portion 211 of the pedestal 21 to be urged to move vertically and swing and used in the therapy area during a beauty therapy course; in this embodiment, the therapy element 30 is in a light energy type, such as laser, infrared, or LED, or in an air-flow energy type, to irradiate light energy on skin or push specific particles into the skin, having spot removal, fur removal, wrinkles removal, rejuvenation, or anti-aging therapy; the controlling device 40 includes a controller 41 electrically connecting with the displacement diving device 20 and the therapy device, and the controller 41 includes an operation panel 42 to control the displacement driving device 20 to urge the therapy element 30 of the therapy device so that therapy element 30 is urged to move straightly or curvedly in the first-axis, the second-axis, and the third-axis directions, and the operation panel 42 is served to set the therapy area, a parameter of energy of the therapy element 30 (i.e., a strength, a spot size, a frequency of a pulse width of the light energy type of therapy element or an output pressure, a speed, a cross-sectional area of the air-flow energy type of therapy element), a therapy distance between the therapy element 30 and the therapy area and a therapy temperature in the therapy area 61; the therapy element 30 of the therapy device includes a first sensor 43 to sense a distance between the therapy element 30 and the therapy area and a second sensor 44 to sense the therapy temperature, the first sensor 43 and the second sensor 44 output a sensed distance between the therapy element 30 and the therapy area and a sensed temperature toward the controller 41 individually so that the operation panel 42 controls the therapy element 30 of the pedestal 21 of the displacement driving device 20 to move straightly or curvedly in the first-axis, the second-axis, and the third-axis directions within the therapy area based on a set therapy parameter, the sensed distance, and the sensed temperature; and during the beauty therapy course, the therapy element 30 is driven to move vertically and swing with a curved profile of the therapy area to have a precise and stable beauty therapy in the therapy area; the supporting member 50 is applied to support the displacement driving device 20 and includes a holder 501 on which an upright stem 502 is secured, and the upright stem 502 includes a horizontal stem 503 fixed on one side thereof to be moved vertically, the horizontal stem 503 utilizes to support the displacement driving device 20, such that the displacement driving device 20 allows movement above the therapy area.

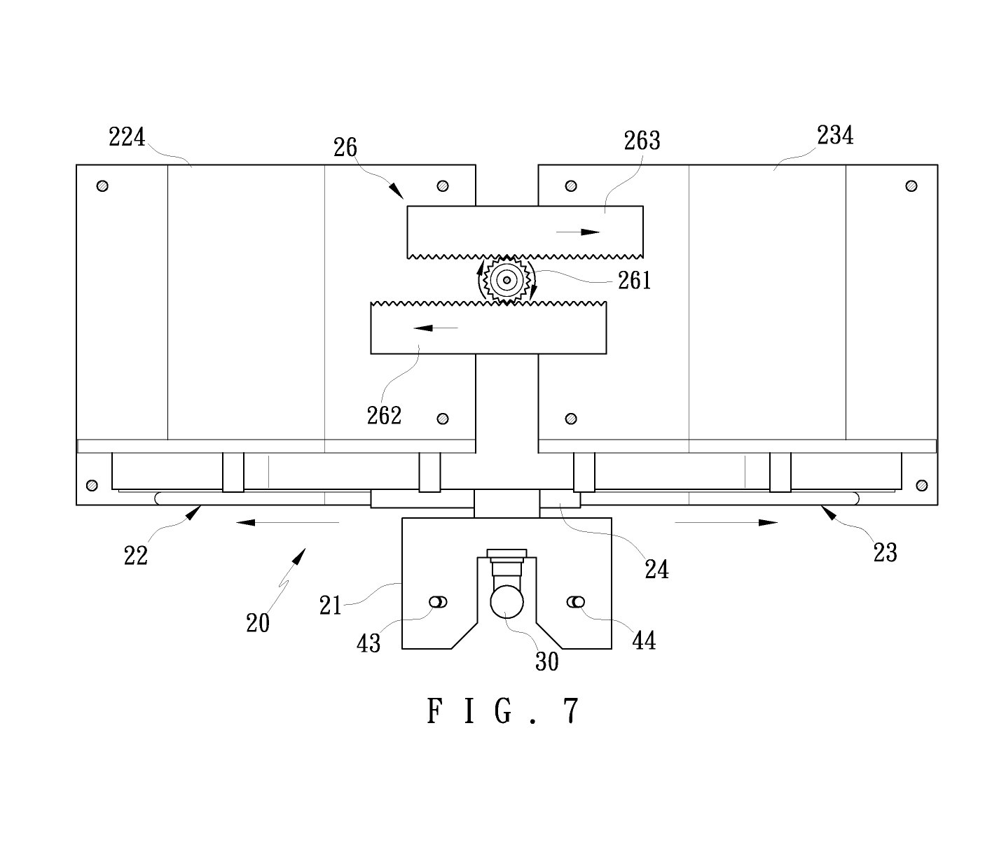

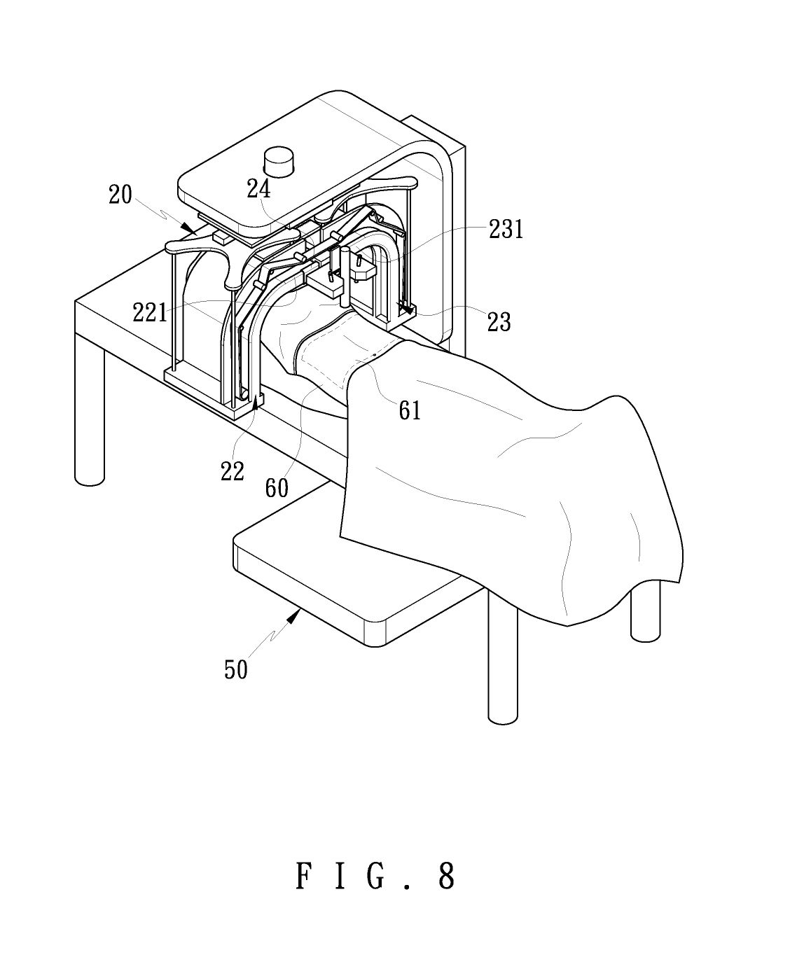

As shown in FIGS. 7 and 8, an automatic controlling method of a beauty therapy, for example, in the beauty therapy for the skin, the displacement driving device 20 is positioned above the therapy area 61 of a human body 60, and the interval width between the left slideway 22 and the right slideway 23 is adjusted according to a profile of the human body 60 so that the first rack 262 and the second rack 263 are adjusted, and the left slideway 22 and the right slideway 23 of the first guiding set are actuated to displace in the first-axis direction, the displacement gap between the first horizontal section 221 of the left slideway 22 and the second horizontal section 231 of the right sideway 23 is used to adjust the left slideway 22 and the right slideway 23 so as to obtain a suitable interval width of the profile of the human body 60, positioning the left slideway 22 and the right slideway 23 above the therapy area 61.

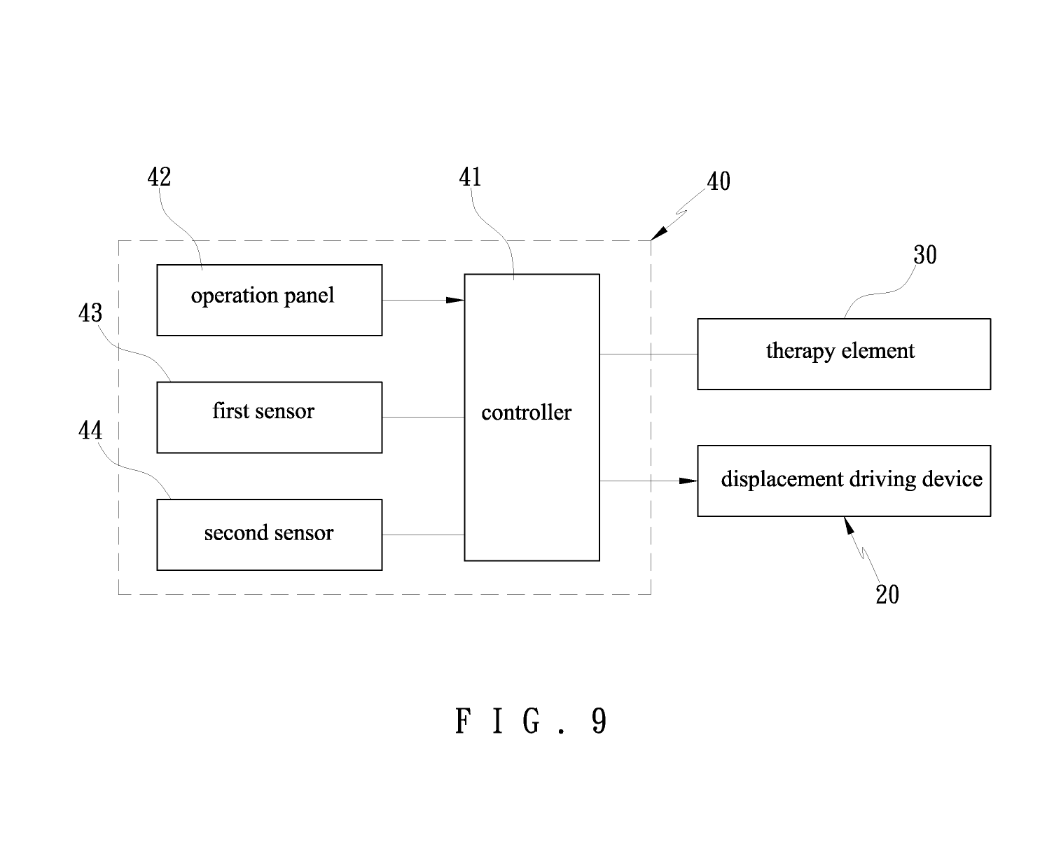

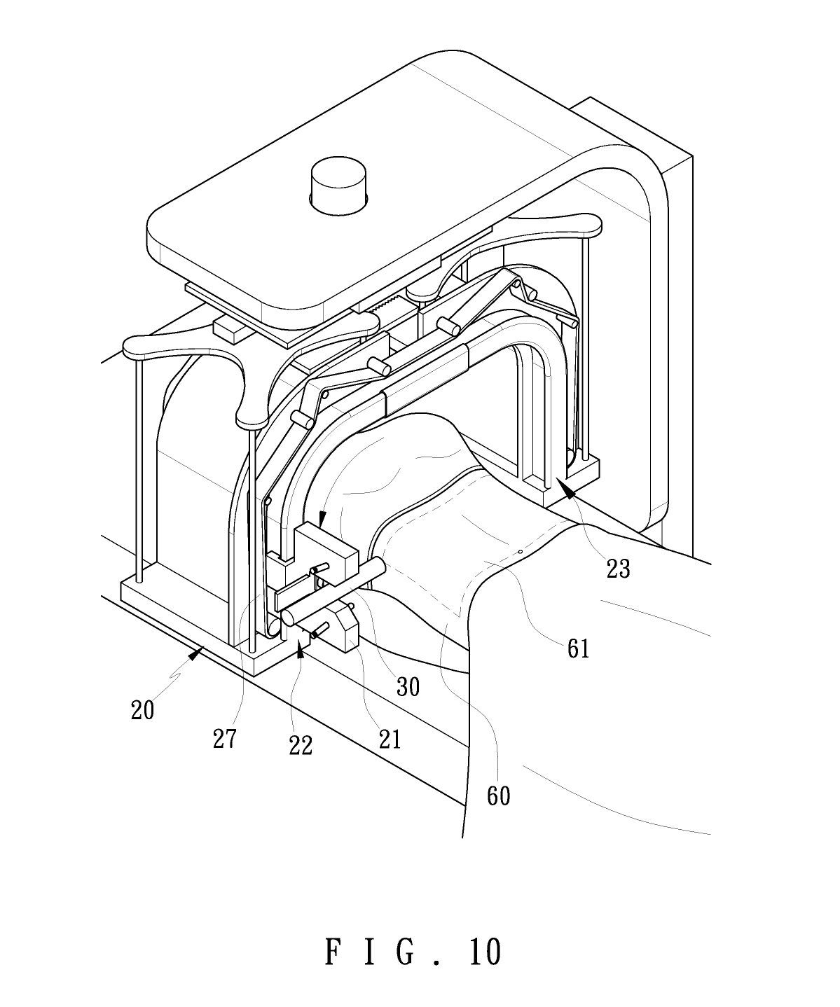

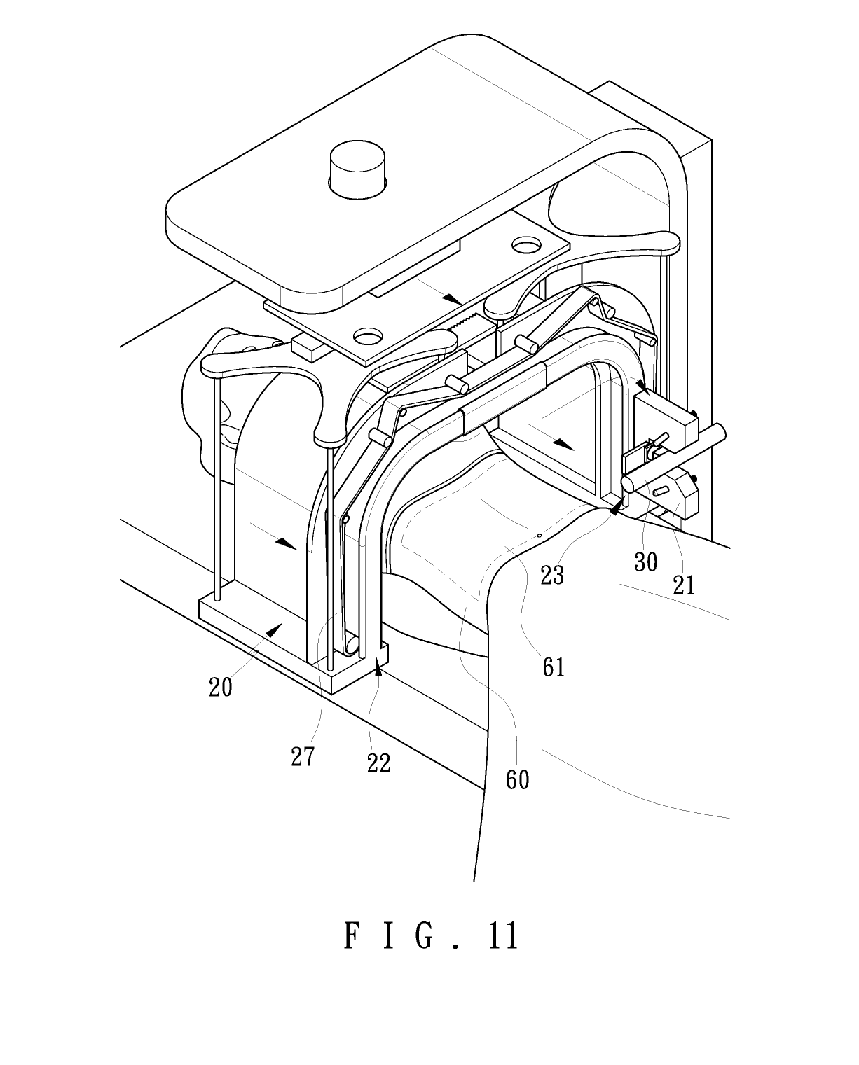

Referring to FIGS. 9-11, during the beauty therapy course, the therapy area 61 of the human body 60 is set by the controlling device 40, and the operation panel 42 of the controlling device 40 is used to control the pedestal 21 to actuate the therapy element 30 so that the therapy element 30 displaces toward a starting position of one side of the human body 60, thus positioning a starting point of a movement path of the therapy element 30. Thereafter, the pedestal 21 of the displacement driving device 20 is controlled to actuate the therapy element 30 to displace toward a stopping position of another side of the human body 60, thereby positioning a stopping point of the movement path of the therapy element 30. After the therapy area 61 of the human body 60 is set, the pedestal 21 is controlled to actuate the therapy element 30 to return back to the starting point; and the operation panel 42 is applied to set the parameter of energy of the therapy element 30 (i.e., a strength, a spot size, a frequency of a pulse width of a laser therapy element), a therapy distance between the therapy element 30 and the therapy area 61, and a therapy temperature of the therapy area 61.

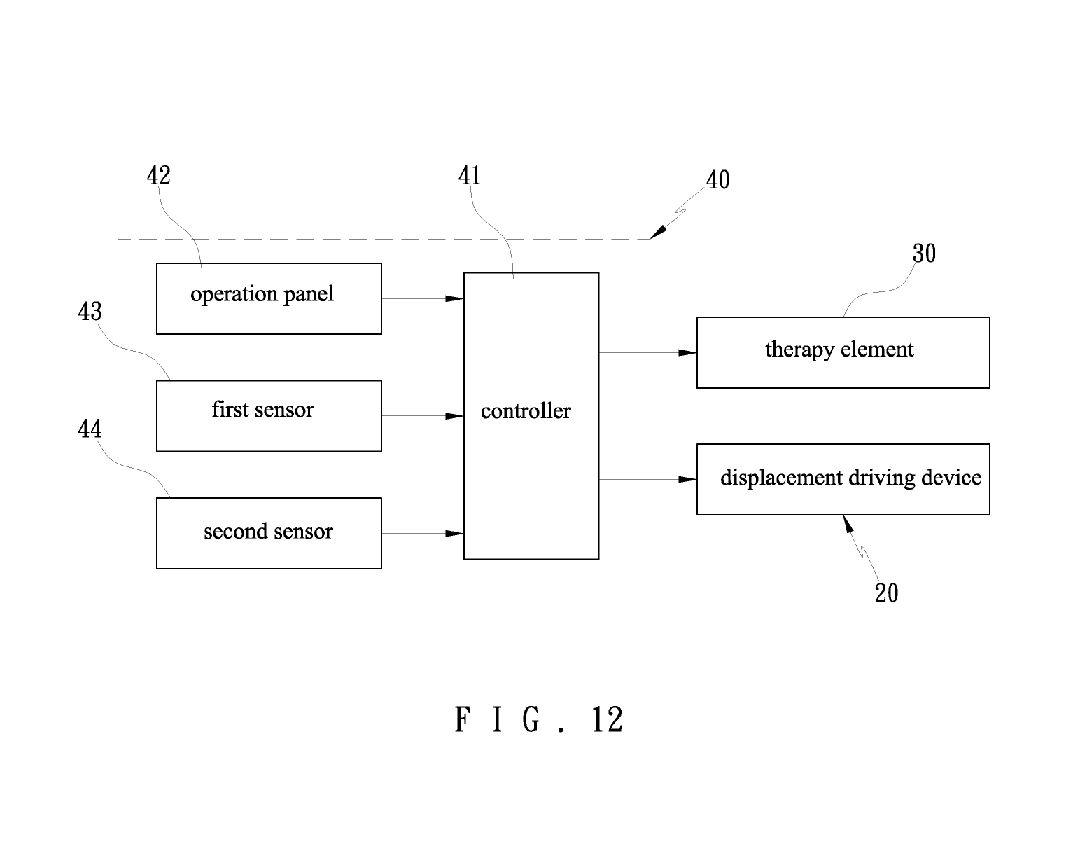

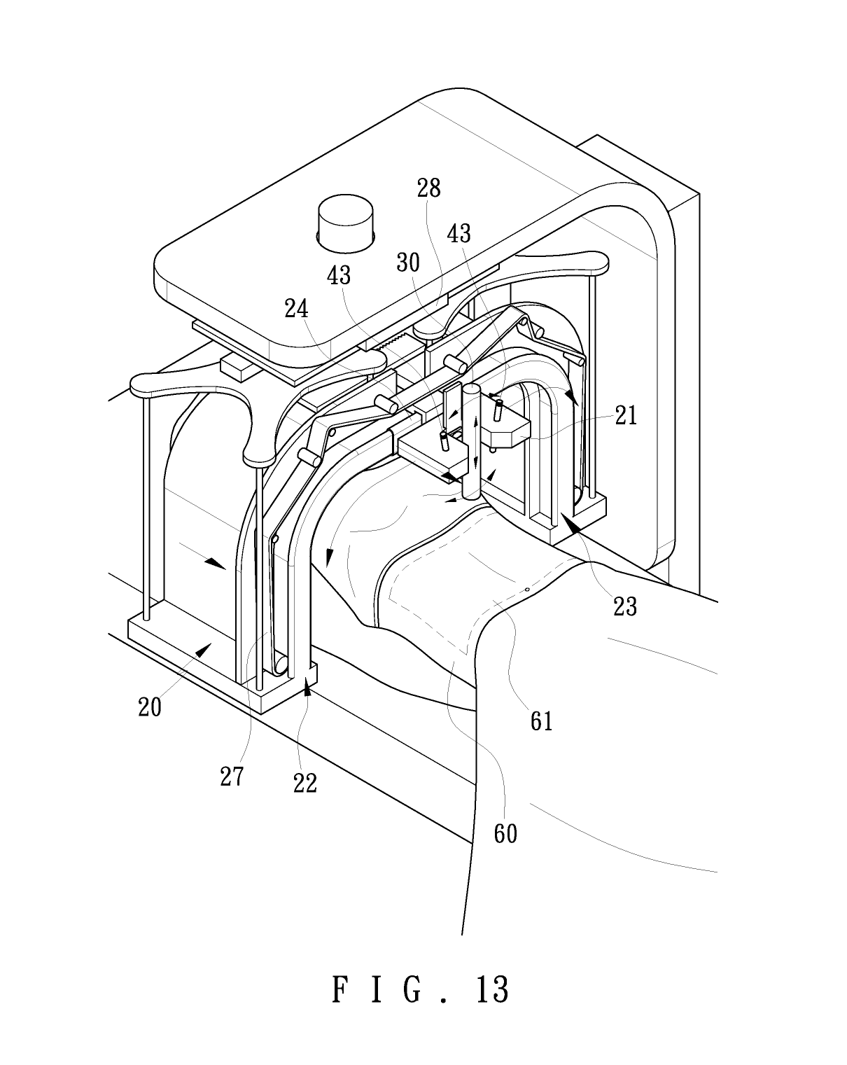

As illustrated in FIGS. 12 and 13, after setting the parameter of the therapy element 30, the displacement driving device 20 and the therapy device are stared so that the controller 41 of the controlling device 40 controls the therapy element 30 to move straightly or curvedly along the left slideway 22 and the right slideway 23 in the first-axis direction and the second-axis direction and to move straightly along the vertical slideway 28 of the second guiding set in the third-axis direction according to the parameter of the energy, the therapy distance, and the therapy temperature, such that the therapy element 30 allows a straight or a curved displacement in the therapy area 61 of the human body 60, having the beauty therapy course. In addition, when the therapy element 30 is driven during the beauty therapy course, the controller 41 receives the sensed distance sensed by the first sensor 43 and the sensed temperature sensed by the second sensor 44, and as a difference occurs between the sensed distance and a set value, the controller 41 controls the therapy element 30 to move vertically and swing based on a received signal so that the therapy element 30 keeps a suitable therapy distance from the therapy area 61. Furthermore, when another difference occurs between the sensed temperature and another set value, the controller 41 controls the displacement driving device 20 and the therapy device to stop operation so as to prevent the human body 60 from burn or a poor therapy.

Thereby, the therapy element 30 can be operated precisely and stably in the therapy area 60 to achieve a therapy effect well.

The invention is not limited to the above embodiment but various modifications thereof may be made. It will be understood by those skilled in the art that various changes in form and detail may made without departing from the scope and spirit of the present invention.

What is claimed is:

1. An automatic beauty therapy equipment comprising:
a displacement driving device including a pedestal to be driven by a displacing mechanism so as to move straightly or curvedly in a first-axis direction, a second-axis direction, and a third-axis direction;
a therapy device including a therapy element fixed in the pedestal of the displacement driving device;
a controlling device including a controller electrically connecting with the displacement diving device and the therapy device, and the controller being used to control the displacement driving device to urge the therapy element of the therapy device to move straightly or curvedly within a therapy area in the first-axis, the second-axis, and the third-axis directions, and the controller being served to set a therapy area so that the therapy element is driven to move vertically and swing with a curved profile of the therapy area to have a precise and stable beauty therapy in the therapy area.

2. The automatic beauty therapy equipment as claimed in claim 1, wherein the pedestal of the displacement driving device includes a receiving portion disposed on one side thereof to receive the therapy element and an actuating block mounted on another side thereof and connected onto the displacing mechanism.

3. The automatic beauty therapy equipment as claimed in claim 1, wherein the displacing mechanism of the displacement driving device includes a first guiding set and a second guiding set, wherein the first guiding set is used to drive the pedestal to displace straightly or curvedly in the first-axis direction and the second-axis direction, and the second guiding set is applied to drive the pedestal to move straightly in the third-axis direction.

4. The automatic beauty therapy equipment as claimed in claim 3, wherein the first guiding set includes a left slideway and a right sideway, both of which include a first horizontal section and a second horizontal section extending in the first-axis direction respectively and a first vertical section and a second vertical section extending in the second-axis direction individually, and between the first horizontal section and the first vertical section is defined a first arcuate section, between the second horizontal section and the second vertical section is defined a second arcuate section.

5. The automatic beauty therapy equipment as claimed in claim 4, wherein between the left slideway and the right slideway is defined an adjusting structure to adjust an interval width between the left slideway and the right slideway; the adjusting structure includes a connecting rail fixed on a connection of the first horizontal section of the left slideway and the second horizontal section of the right sideway, the left slideway and the right sideway are joined with a slidable rail assembly so as to move in the first-axis direction, and between the left slideway and the right slideway is defined an actuation assembly to drive the left slideway and the right slideway to displace in the first-axis direction respectively so that the interval width between the left slideway and the right slideway is adjusted.

6. The automatic beauty therapy equipment as claimed in claim 5, wherein an actuation assembly includes a gear fixed therein to be driven to rotate, and two sides of the gear engage with a first rack of the first side plate and a second rack of the second side plate respectively.

7. The automatic beauty therapy equipment as claimed in claim 3, wherein the second guiding set of the displacement driving includes a vertical slideway extending therefrom in the third-axis direction.

8. The automatic beauty therapy equipment as claimed in claim 1, wherein and the controlling device includes an operation panel to set a therapy area, a parameter of energy of the therapy element, a therapy distance between the therapy element and the therapy area, and a therapy temperature in the therapy area.

9. The automatic beauty therapy equipment as claimed in claim 1, wherein the therapy device includes a first sensor to sense a distance between the therapy element and the therapy area and a second sensor to sense the therapy temperature in the therapy area, and the first sensor and the second sensor output a sensed distance between the therapy element and the therapy area and a sensed temperature in the therapy area toward the controller individually.

10. The automatic beauty therapy equipment as claimed in claim 9, wherein the controller controls the therapy element of the pedestal of the displacement driving device to move straightly or curvedly in the first-axis, the second-axis, and the third-axis directions within the therapy area based on a set therapy parameter, the sensed distance, and the sensed temperature, and during a beauty therapy course, the therapy element is driven to move vertically and swing with a curved profile of the therapy area.
